(19) United States
(12) Patent Application Publication
(10) Pub. No.: US 2026/0224468 A1
(43) Pub. Date: Aug. 6, 2026

(54) NUTRIENT-BASED TEMPORARY TATTOO INK COMPOSITION

(71) Applicant: Jake BAIR, Buffalo, NY (US)

(72) Inventor: Jake BAIR, Buffalo, NY (US)

(21) Appl. No.: 19/044,823

(22) Filed: Feb. 4, 2025

(51) Int. Cl. A61K 8/9789; A61Q 1/02

(52) U.S. Cl. CPC A61K 8/9789; A61Q 1/025; A61K 2800/432; A61K 2800/48; A61K 2800/592

(57) ABSTRACT

A nutrient-based temporary tattoo ink composition comprising a liquid base, a solid component selected for its ability to be absorbed into a body through skin, a plant-based color pigment, and a thickening agent.

### FIELD OF THE INVENTION

The present invention relates generally to tattoo inks. In particular, the present invention relates to a nutrient-based temporary tattoo ink composition.

### BACKGROUND OF THE INVENTION

Humans have used ink to mark their skin for thousands of years. These markings, or tattoos, have been discovered on mummified human skin dating back to the 4^{th} millennium BCE. The oldest known sample of tattooed skin was found on the body of Otzi the Iceman, dating to between 3370 and 3100 BCE. Archaeologists have also discovered possible tattooing tools that would suggest the practice of tattooing was being performed far before the time period when Otzi was believed to be alive.

Traditionally, tattoos are created by injecting pigment into the skin's dermis, the layer of dermal tissue underlying the epidermis. The pigment is then dispersed throughout a homogenized damaged layer of the epidermis and upper dermis, in both of which the presence of the tattoo ink activates the body's immune system's phagocytes to engulf the pigment particles. As the body heals, the damaged epidermis flakes away while skin granulation tissue forms deeper within the skin, which is later converted to connective tissue by collagen growth. This action heals the upper dermis, where the pigment remains trapped within fibroblasts, with the pigment ultimately concentrating in a layer just below the dermis/epidermis boundary. While tattoos produced by this method tend to degrade over time as the pigment migrates deeper into the dermis, the tattoo is generally permanent and will remain visible throughout the life of the tattooed individual.

Today, tattoos have become commonplace among the general population. A recent Pew Research Center study found that 32% of U.S. adults have a tattoo, and 22% of U.S. adults have multiple tattoos. As tattoos are becoming more popular, people are looking for ways to have tattoos that last varying amounts of time, and which are not permanent. For example, U.S. Pat. No. 6,013,222 discloses a tattoo ink comprised of pigments or dyes combined with a tattoo pigment vehicle associated with the pigment. The tattoo pigment vehicles can be formulated so that they remain indefinitely in the dermis, they can spontaneously disappear after a predetermined period of time, or they can be caused to disappear by the imposition of an exogenous force, such as thermal, sonic, light, laser, or infrared forces.

With the rapid increase of people who have, or would like to have, tattoos, the reasons for receiving tattoos have also expanded. For much of history, people have been receiving tattoos for their ornamental aspect related to artistic self-expression. Tattoos were, and still are, used to commemorate important moments, loved ones, or cultural pride, as well as for other non-commemorative aspects of self-expression. Today, however, tattoos are used for more than just self-expression. Tattoos can be used for permanent cosmetics such as eyeliner or lip color. Tattoos can also be used for medical applications, such as being applied following breast reconstruction following a mastectomy or to treat hypo-and hyper-pigmentation caused by a variety of dermatological conditions. Tattoos are being implemented more frequently for these types of medical uses, and as the general population becomes more accustomed to tattoos, the medical uses of tattoos will continue to expand.

It is well known in the art to apply and absorb medications through the skin. The most common route of medication absorption through the skin, i.e., transdermal medications, is through passive diffusion. Passive diffusion through the skin is a process where the medication moves from areas of higher concentrations to areas of lower concentrations across the layers of the skin, where the medication is eventually absorbed into the bloodstream. However, the majority of biomolecules cannot penetrate the skin barrier, particularly the epidermis. For a molecule to cross the epidermis and enter into the dermis where it can be absorbed into the bloodstream, the molecule must possess properties similar to the skin's surface, i.e., it must be lipophilic, have a slightly acidic pH (between 4.6 and 5.5), and have a molecular wight smaller than 500 Daltons.

Therefore, there is a long-felt need for a nutrient-based temporary tattoo ink that can be absorbed through the skin into the body. More specifically, there is a long felt need for a nutrient-based temporary tattoo ink that as the body absorbs the nutrient-based ink, the tattoo will begin to fade at different rates, depending on the concentrations of pigments and nutrients within the ink.

### SUMMARY

In a first embodiment of the present invention, the present invention broadly comprises a nutrient-based temporary tattoo ink composition comprising a liquid base, a solid component selected for its ability to be absorbed into a body through skin, a plant-based color pigment, and a thickening agent.

In one aspect of the first embodiment, the present invention comprises the aforementioned nutrient-based temporary tattoo ink composition, wherein the plant-based color pigment is derived from beetroot.

In another aspect of the first embodiment, the present invention comprises the aforementioned nutrient-based temporary tattoo ink composition, wherein the plant-based color pigment is derived from turmeric.

In another aspect of the first embodiment, the present invention comprises the aforementioned nutrient-based temporary tattoo ink composition, wherein the plant-based color pigment is derived from spinach.

In another aspect of the first embodiment, the present invention comprises the aforementioned nutrient-based temporary tattoo ink composition, wherein the thickening agent is comprised of xanthan gum.

In another aspect of the first embodiment, the present invention comprises the aforementioned nutrient-based temporary tattoo ink composition, wherein the tattoo ink composition has a concentration of the liquid base from 32% to 42% by weight, a concentration of the solid component from 15% to 25% by weight, a concentration of the plant-based color pigment from 20% to 30% by weight, and a concentration of the thickening agent from 11% to 21% by weight.

In a second embodiment of the present invention, the present invention comprises a nutrient-based temporary tattoo ink composition comprising a liquid base comprising water, a hyaluronic acid, an aloe vera gel, and a carrier oil blend; at least one solid component selected for its ability to be absorbed into a body through skin, the solid components comprising at least one of collagen powder, vitamin C (ascorbic acid) powder, vitamin E (tocopherol) oil, chamomile extract, calendula extract, zinc oxide, and green tea extract; at least one plant-based color pigment; and a thickening agent.

In one aspect of the second embodiment, the present invention comprises the aforementioned nutrient-based temporary tattoo ink composition, wherein the plant-based color pigment is derived from beetroot.

In another aspect of the second embodiment, the present invention comprises the aforementioned nutrient-based temporary tattoo ink composition, wherein the plant-based color pigment is derived from turmeric.

In another aspect of the second embodiment, the present invention comprises the aforementioned nutrient-based temporary tattoo ink composition, wherein the plant-based color pigment is derived from spinach.

In another aspect of the second embodiment, the present invention comprises the aforementioned nutrient-based temporary tattoo ink composition, wherein the hyaluronic acid comprises a humectant.

In another aspect of the second embodiment, the present invention comprises the aforementioned nutrient-based temporary tattoo ink composition, wherein the carrier oil blend is comprised of a vegetable oil.

In another aspect of the second embodiment, the present invention comprises the aforementioned nutrient-based temporary tattoo ink composition, wherein the carrier oil blend is comprised of coconut oil.

In another aspect of the second embodiment, the present invention comprises the aforementioned nutrient-based temporary tattoo ink composition, wherein the carrier oil blend is comprised of jojoba oil.

In another aspect of the second embodiment, the present invention comprises the aforementioned nutrient-based temporary tattoo ink composition, wherein the thickening agent is comprised of xanthan gum.

In another aspect of the second embodiment, the present invention comprises the aforementioned nutrient-based temporary tattoo ink composition, wherein the tattoo ink composition has a concentration of the liquid base from 32% to 42% by weight, a concentration of the solid components from 15% to 25% by weight, a concentration of the plant-based color pigment from 20% to 30% by weight, and a concentration of the thickening agent from 11% to 21% by weight.

In another aspect of the second embodiment, the present invention comprises the aforementioned nutrient-based temporary tattoo ink composition, wherein the concentration of the solid components has a concentration of the collagen powder from 2.5% to 4.5% by weight, the vitamin C (ascorbic acid) powder from 2.5% to 4.5% by weight, the vitamin E (tocopherol) oil from 3% to 4% by weight, the chamomile extract from 1% to 3% by weight, the calendula extract from 1% to 3% by weight, the zinc oxide from 4% to 6% by weight, and the green tea extract from 1% to 3% by weight.

A general object of the present invention is to provide a nutrient-based temporary tattoo ink which can absorb into a body through skin over time. The tattoo ink is applied like a traditional tattoo using a tattoo machine. Over time, the body gradually breaks down the components of the ink, enabling the body to use the components to promote healing processes, reduce inflammation, or improve immune function.

Another object of the present invention is to allow a tattoo applied to the skin the ability to fade at different rates over time depending on the concentrations of pigments and nutrients within the nutrient-based temporary tattoo ink.

These and other objects, features, and advantages of the present invention will become readily apparent upon a review of the following detailed description of the invention, in view of the appended claims.

### DETAILED DESCRIPTION

At the outset, it should be understood that this disclosure is not limited to the particular methodology, materials and modifications described and as such may, of course, vary. It is also understood that the terminology used herein is for the purpose of describing particular aspects only, and is not intended to limit the scope of the claims. Those in the art will understand that any suitable material, now known, or hereafter developed, may be used in forming the present invention described herein.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be noted that the terms “including,” “includes,” “having,” “has,” “contains,” and/or “containing,” should be interpreted as being substantially synonymous with the terms “comprising” and/or “comprises.”

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” or “one of:” is used herein.

Generally, the present invention is related to nutrient-based temporary tattoo ink comprised of a liquid base, solid components selected for their ability to be absorbed into a human body through the skin, plant-based color pigments, and thickening agents. The liquid base, solid components, plant-based color pigments, and thickening agents are formulated in such a way as to allow the body to break down and absorb each component, and in turn allow for a tattoo created through the use of the ink to be temporary. The concentration of each component of the ink can provide shorter or longer time frames with regard to how long the tattoo will remain visible in the skin. Further, the nutrient-based temporary tattoo ink will be fully innocuous and biologically tolerable to the user.

The solid components will have the physical and chemical characteristics necessary to be absorbed into the bloodstream of the user through the user's skin. The solid components, among other uses, are comprised of nutrients that the body can use to promote healing processes, reduce inflammation, improve immune function, and improve the overall wellbeing of the user.

In order to produce temporary tattoos, the pigment should be a natural plant-based pigment and have a particle size small enough to be absorbed by the body.

### Solid Components

The invention relates to the use of solid components that can be absorbed through the skin into the bloodstream of the user, where they can be further utilized by the body. The solid components are comprised of nutrients and other natural materials which can be gradually broken down by the body after the application of the tattoo ink into the skin.

The solid components may comprise any substance that is biologically tolerated and can be absorbed through the skin into the bloodstream. These solid components may be, but are not limited to, collagen powder, vitamin C (ascorbic acid) powder, vitamin E (tocopherol) oil, chamomile extract, calendula extract, zinc oxide, or green tea extract.

### Pigments

The invention relates to the use of natural plant-based color pigments that have a particle size small enough to be broken down and absorbed by the body over time. Like the solid components, the plant-based color pigments may comprise any substance that is biologically tolerated and can be absorbed through the skin into the body. The plant-based color pigments may be derived from, but are not limited to, beetroot, turmeric, or spinach.

### Liquid Base

The invention relates to the use of a liquid base which forms the foundation for the nutrient-based temporary tattoo ink. The liquid base facilitates the mixing of the components of the ink, and also ensures uniform distribution and dissolution of the remaining components.

In one embodiment, the liquid base is prepared by combining water, hyaluronic acid, aloe vera gel, and a carrier oil blend. In a preferred embodiment, the hyaluronic acid acts as a humectant. The hyaluronic acid may be, but is not limited to, sodium hyaluronate, hydrolyzed hyaluronic acid, or a sodium hyaluronate cross polymer. Further, the carrier oil blend of the liquid base may by, but is not limited to, vegetable oils such as coconut oil or jojoba oil.

### Additives

An additive will be all of the components forming part of the nutrient-based temporary tattoo ink composition which are intended to help stabilize, mix, emulsify and preserve the components of the tattoo ink. In a preferred embodiment, an example of an additive is a thickening agent comprised of xanthan gum. Further examples of additives may by preservatives which may be, but are not limited to, vitamin C and vitamin E.

### Process

The above objects are achieved by a method of preparing the nutrient-based temporary tattoo ink, which is characterized generally by the following processes:

### 1. Ingredient Preparation

a. Ensure all components are pure and free from contamination.
b. Determine optimal concentrations of each component based on its desired therapeutic effects, compatibility with the user, and safety considerations.
c. Prepare the liquid base by combining water, hyaluronic acid, aloe vera gel, and carrier oil.
d. Incorporate the solid components into the liquid base, ensuring uniform distribution and dissolution.
e. Incorporate the natural plant-based pigments to achieve the desired color of the ink.
f. Incorporate thickeners, for example xanthan gum, to achieve the desired consistency for applying a tattoo. The ink should have an optimal viscosity for being contained in and used with a tattoo machine, as well as for maintaining stability during application.
e. Adjust the pH of the ink to a level that is compatible with a user's skin pH.

### 2. Mixing and Homogenization

a. Use of mechanical homogenization or other mixing equipment to ensure thorough blending of all ingredients.
b. Perform quality control tests, for example, viscosity measurements, pH measurements, and stability assessments.

### 3. Sterilization and Safety Measures

a. Implement a sterilization process for the ink to eliminate microbial contaminates. The sterilization process may include, but is not limited to, filtration or heat treatments.
b. Package the ink in sterile containers to maintain purity. The packaging may be single use.
c. Conduct safety and efficacy testing through in vitro and in vivo testing, ensuring the ink meets any regulatory standards and does not cause adverse effects.

With the above-described preparation, a nutrient-based temporary tattoo ink is obtained, which comprises natural components which can be absorbed through the skin into the body of user, and which fades over time as the body breaks down and absorbs the components.

### EXAMPLES

The present invention shall specifically be explained below with reference to examples, but the present invention shall not be restricted by these examples. In the examples, % shows % by weight of the ink composition.

### Example 1

### Liquid Base:

Hyaluronic Acid Solution
   7%
Aloe Vera Gel
   15%
Coconut oil
   15%

sSolid Components:
Collagen Powder
   3.5%
Vitamin C (Ascorbic Acid) Powder
   3.5%
Vitamin E (Tocopherol) Oil
   4%
Chamomile Extract (Concentrated)
   2%
Calendula Extract (Concentrated)
   2%
Zinc Oxide
   5%
Green Tea Extract (Concentrated)
   2%

### Pigments:

Natural Plant-Based Pigment Derived from Beetroot
   25%

### Thickener:

Xanthan Gum
   16%

A nutrient-based temporary tattoo ink composition was obtained comprising the above components using the method of preparation as described herein.

### Example 2

### Liquid Base:

Hyaluronic Acid Solution
   5%
Aloe Vera Gel
   12%
Coconut oil
   16%

### Solid Components:

Collagen Powder
   4%
Vitamin C (Ascorbic Acid) Powder
   4%
Vitamin E (Tocopherol) Oil
   4%
Chamomile Extract (Concentrated)
   3%
Calendula Extract (Concentrated)
   3%
Zinc Oxide
   4%
Green Tea Extract (Concentrated)
   2%

### Pigments:

Natural Plant-Based Pigment Derived from Turmeric
   29%

### Thickener:

Xanthan Gum
   14%

A nutrient-based temporary tattoo ink composition was obtained comprising the above components using the method of preparation as described herein.

### Example 3

### Liquid Base:

Hyaluronic Acid Solution
   7%
Aloe Vera Gel
   20%
Jojoba oil
   15%

### Solid Components:

Collagen Powder
   6%

### Pigments:

Natural Plant-Based Pigment Derived from Spinach
   36%

### Thickener:

Xanthan Gum
   16%

A nutrient-based temporary tattoo ink composition was obtained comprising the above components using the method of preparation as described herein.

### Example 4

### Liquid Base:

Hyaluronic Acid Solution
   7%
Aloe Vera Gel
   15%
Jojoba oil
   15%

### Solid Components:

Collagen Powder
   3.5%
Vitamin C (Ascorbic Acid) Powder
   3.5%
Vitamin E (Tocopherol) Oil
   4%
Chamomile Extract (Concentrated)
   2%
Calendula Extract (Concentrated)
   2%
Zinc Oxide
   5%
Green Tea Extract (Concentrated)
   2%

### Pigments:

Natural Plant-Based Pigment Derived from Turmeric
   29%

### Thickener:

Xanthan Gum
   12%

A nutrient-based temporary tattoo ink composition was obtained comprising the above components using the method of preparation as described herein.

The embodiments described are merely exemplary and various alternatives, combinations, omissions of specific components, or foreseeable alternative components, understood by one having ordinary skill in the art, described in the present disclosure or within the field of the present disclosure, are intended to fall within the scope of the appending claims.

It will be appreciated that various aspects of the invention and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the claims.

What is claimed is:

1. A nutrient-based temporary tattoo ink composition, comprising:
a liquid base;
a solid component selected for its ability to be absorbed into a body through skin;
a plant-based color pigment; and,
a thickening agent.

2. The nutrient-based temporary tattoo ink composition recited in claim 1, wherein said plant-based color pigment is derived from beetroot.

3. The nutrient-based temporary tattoo ink composition recited in claim 1, wherein said plant-based color pigment is derived from turmeric.

4. The nutrient-based temporary tattoo ink composition recited in claim 1, wherein said plant-based color pigment is derived from spinach.

5. The nutrient-based temporary tattoo ink composition recited in claim 1, wherein said thickening agent is comprised of xanthan gum.

6. The nutrient-based temporary tattoo ink composition recited in claim 1, wherein said tattoo ink composition has a concentration of said liquid base from 32% to 42% by weight, a concentration of said solid component from 15% to 25% by weight, a concentration of said plant-based color pigment from 20% to 30% by weight, and a concentration of said thickening agent from 11% to 21% by weight.

7. A nutrient-based temporary tattoo ink composition, comprising:
a liquid base comprising:
water;
a hyaluronic acid;
an aloe vera gel; and,
a carrier oil blend;
at least one solid component selected for its ability to be absorbed into a body through skin, said solid components comprising at least one of collagen powder, vitamin C (ascorbic acid) powder, vitamin E (tocopherol) oil, chamomile extract, calendula extract, zinc oxide, and green tea extract;
at least one plant-based color pigment; and,
a thickening agent.

8. The nutrient-based temporary tattoo ink composition recited in claim 7, wherein said plant-based color pigment is derived from beetroot.

9. The nutrient-based temporary tattoo ink composition recited in claim 7, wherein said plant-based color pigment is derived from turmeric.

10. The nutrient-based temporary tattoo ink composition recited in claim 7, wherein said plant-based color pigment is derived from spinach.

11. The nutrient-based temporary tattoo ink composition recited in claim 7, wherein said hyaluronic acid comprises a humectant.

12. The nutrient-based temporary tattoo ink composition recited in claim 7, wherein said carrier oil blend is comprised of a vegetable oil.

13. The nutrient-based temporary tattoo ink composition recited in claim 7, wherein said carrier oil blend is comprised of coconut oil.

14. The nutrient-based temporary tattoo ink composition recited in claim 7, wherein said carrier oil blend is comprised of jojoba oil.

15. The nutrient-based temporary tattoo ink composition recited in claim 7, wherein said thickening agent is comprised of xanthan gum.

16. The nutrient-based temporary tattoo ink composition recited in claim 7, wherein said tattoo ink composition has a concentration of said liquid base from 32% to 42% by weight, a concentration of said solid components from 15% to 25% by weight, a concentration of said plant-based color pigment from 20% to 30% by weight, and a concentration of said thickening agent from 11% to 21% by weight.

17. The nutrient-based temporary tattoo ink composition recited in claim 16, wherein said concentration of said solid components has a concentration of:
said collagen powder from 2.5% to 4.5% by weight;
said vitamin C (ascorbic acid) powder from 2.5% to 4.5% by weight;
said vitamin E (tocopherol) oil from 3% to 4% by weight;
said chamomile extract from 1% to 3% by weight;
said calendula extract from 1% to 3% by weight;
said zinc oxide from 4% to 6% by weight; and,
said green tea extract from 1% to 3% by weight.
